(19) United States
(12) Patent Application Publication
(10) Pub. No.: US 2005/0054587 A1
(43) Pub. Date: Mar. 10, 2005

(54) Novel therapeutic indication of azithromycin for treatment of non-infective inflamatory diseases

(76) Inventors: Ognjen Culic, Zagreb (HR); Michael Parnham, Zagreb (HR); Vesna Erakovic, Rijeka (HR)

Correspondence Address: DARBY & DARBY P.C., P. O. BOX 5257, NEW YORK, NY, 10150-5257, US

(21) Appl. No.: 10/476,377

(22) PCT Filed: Apr. 10, 2002
(86) PCT No.: PCT/EP02/03984

(30) Foreign Application Priority Data
Apr. 27, 2001 (HR) P010301A

(51) Int. Cl. A61K 31/7052

(52) U.S. Cl. 514/28

(57) ABSTRACT

The invention relates to the use of 9-deoxo-9-dihydro-9a-methyl-9a-homoerythromycin. A (generic name: azithromycin) for the therapy of neutrophil-dominated non-infective inflammatory diseases, pharmaceutical compositions containing azithromycin for enteral or parenteral administration and methods for the production of these pharmaceutical compositions.

The invention relates to the use of 9-deoxo-9-dihydro-9a-methyl-9a-aza-9a-homoerythromycin A (generic name: azithromycin) for the therapy of neutrophil-dominated non-infective inflammatory diseases, pharmaceutical compositions containing azithromycin for enteral or parenteral administration and methods for the production of these pharmaceutical compositions.

Most inflammatory diseases are characterised by abnormal accumulation of inflammatory cells including monocytes/macrophages, granulocytes, plasma cells, lymphocytes and platelets. Along with tissue endothelial cells and fibroblasts, these inflammatory cells release a complex array of lipids, growth factors, cytokines and destructive enzymes that cause local tissue damage.

One form of inflammatory response is neutrophilic inflammation which is characterized by infiltration of the inflamed tissue by neutrophil polymorphonuclear leucocytes (PMN), which are a major component of host defence. Tissue infection by extracellular bacteria represents the prototype of this inflammatory response. On the other hand, various non-infectious diseases are characterized by extravascular recruitment of neutrophils. This group of inflammatory diseases includes chronic obstructive pulmonary disease, adult respiratory distress syndrome, some types of immune-complex alveolitis, cystic fibrosis, bronchitis, bronchiectasis, emphysema, glomerulonephritis, active phases of rheumatoid arthritis, gouty arthritis, ulcerative colitis, certain dermatoses such as psoriasis and vasculitis. In these conditions neutrophils are thought to play a crucial role in the development of tissue injury which, when persistent, can lead to the irreversible destruction of the normal tissue architecture with consequent organ dysfunction. Thereby tissue damage is mainly caused by the activation of neutrophils followed by their release of proteinases and increased production of oxygen species.

Chronic obstructive pulmonary disease (COPD) is basically a condition described by the progressive development of airflow limitation that is not fully reversible (ATC, 1995). Most patients with COPD have three pathological conditions: bronchitis, emphysema and mucus plugging. This disease is characterised by a slowly progressive and irreversible decrease in forced expiratory volume in the first second of expiration (FEV₁), with relative preservation of forced vital capacity (FVC) (Barnes, N. Engl. J. Med. (2000), 343(4): 269-280). In both asthma and COPD there is significant, but distinct, remodelling of airways. Most of the airflow obstruction is due to two major components, alveolar_destruction (emphysema) and small airways obstruction (chronic obstructive bronchitis). In COPD it is mainly characterised by profound mucus cell hyperplasia.

Cigarette smoking, air pollution and other environmental factors are major causes of the disease. The causal mechanism remains currently undefined but oxidant-antioxidant disturbances are strongly implicated in the development of the disease. COPD is a chronic inflammatory process that differs markedly from that seen in asthma, with different inflammatory cells, mediators, inflammatory effects and responses to treatment (Keatings et al., Am. J. Respir. Crit. Care Med. (1996), 153: 530-534). Primarily, neutrophil infiltration of the patient's lungs is a characteristic of this disease.

Elevated levels of proinflammatory cytokines like TNF-α, and especially chemokines like IL-8 and GRO-α seem to play a very important role in pathogenesis of this disease. Platelet thromboxane synthesis is also enhanced in patients with COPD (Keatings et al., Am. J. Respir. Crit. Care Med. (1996), 153: 530-534; Stockley and Hill, Thorax (2000), 55(7): 629-630). Most of the tissue damage is caused by activation of neutrophils followed by their release of (metallo)proteinases, and increased production of oxygen species (Repine et al., Am. J. Respir. Crit. Care Med. (1997), 156: 341-357; Barnes, Chest (2000), 117(2 Suppl): 10S-14S).

Most therapeutic endeavour is directed towards the control of symptoms (Barnes, Trends Pharm. Sci. (1998), 19(10): 415-423; Barnes, Am. J. Respir. Crit. Care Med. (1999) 160: 572-S79; Hansel et al., Expert Opin. Investig. Drugs (2000) 9(1): 3-23). Symptoms usually equate with airflow limitation and bronchodilators are the therapy of choice.

Prevention and treatment of complications, prevention of deterioration and improved quality and length of life are also primary goals stated in the three key international guidelines for the management of COPD (Culpitt and Rogers, Exp. Opin. Pharmacother. (2000) 1(5): 1007-1020; Hay, Curr. Opin. Chem. Biol. (2000), 4: 412-419). Basically, most of the current therapeutic research has been focused on mediators involved in the recruitment and activation of neutrophils, or attenuation of consequences of their undesirable activation (Stockley et al., Chest (2000), 117(2 Suppl): 58S-62S).

There are a number of reports on immunomodulatory action of macrolide antibiotics in vitro (Labro, J. Antimicrob. Chemother. (1998), 41 (Suppl B): 37-46; Labro, Clin. Microb. Rev. (2000), 13(4): 615-650; Wales and Woodhead, Thorax (1999), 54 (Suppl 2): S58-S62). Macrolide antibiotics are macrocyclic compounds containing for example a 12-, 14-, 16- or 17-membered lactone ring and 1 to 3 sugar residues, which are linked to each other or to the aglucone by glycosidic bounds. Known members of macrolide antibiotics are for example carbomycin, erythromycin, leucomycin and spiramycin.

The most important findings with regard to macrolide interaction with phagocytic inflammatory cells in vitro concern the inhibitory effects on oxidant production by stimulated cells (Labro et al., J. Antimicrob. Chemother. (1989), 24 (4): 561-572; Umeki, Chest (1993), 104: 1191-1193; Wenisch at al., Antimicrob. Agents Chemother. (1996), 40(9): 2039-2042) and modulation of pro-inflammatory and anti-inflammatory cytokine release by these cells (Labro et al., J. Antimicrob. Chemother. (1989), 24 (4): 561-572; Khan et al, Internat. J. Antimicrob. Agents. (1999), 11: 121-132; Morikawa et al., Antimicrob. Agents and Chemother. (1996), 40(6): 1366-1370; Sugiyama et al., Eur. Respir. J. (1999), 14: 1113-1116). In addition, several macrolides directly stimulate exocytosis (degranulation) by human neutrophils in vitro (Abdelghaffae et al., Antimicrob. Agents Chemother. (1994), 38(7): 1548-1554; Vazifeh et al., Antimicrob. Agents Chemother. (1998), 42 (8): 1944-1951). In the experimental inflammatory model of carrageenin pleurisy in the rat, some macrolide antibiotics like roxithromycin, clarithromycin and erythromycin, but not azithromycin, were found to show anti-inflammatory activity which probably depended on their ability to prevent the production of pro-inflammatory mediators and cytokines. In this model of acute inflammation, NO production, TNF-α levels or PGE₂ were significantly reduced by the antibiotic pre-treatment (Ianario et al., J. Pharmacol. Exp. Ther. (2000), 292: 156-163).

Erythromycin administration also caused anti-inflammatory effects in zymosan-induced peritonitis in rats (Agen et al., Agents Actions (1993), 38(1-2): 85-90). Roxithromycin was reported to be active in reducing the acute inflammatory reaction through a mechanism different from conventional anti-inflammatory agents such as indomethacin. In another study, roxithromycin was demonstrated to be effective in a standard animal model used for evaluating the effects of anti-inflammatory drugs on carrageenin-induced paw oedema, whereas clarithromycin and azithromycin showed modest activity (Scaglione and Rossini, J. Antimicrob. Chemother. (1998), 41, Suppl B: 47-50).

Some macrolide antibiotics, like erythromycin, clarithromycin and roxithromycin have already been used as anti-inflammatory drugs, especially for the treatment of diffuse panbronchiolitis. Reports on the use of macrolides for diseases like rheumatoid arthritis and cystic fibrosis are available (Arayssi et al., Programm and Abstracts of the 4^{th} International conference on macrolides, azalides, streptogramins and ketolides, 21-23 Jan. 1998, Barcelona, Spain, Abstract 6; Singh, J. Assoc. Phys. India (1989), 37: 547; Jaffe et al., Lancet (1998), 351: 420). With regard to relevant pharmacological effects of macrolides, it has been reported that erythromycin inhibits hypersecretion due to inhibition of mucus and water secretion from epithelial cells. It also inhibits neutrophil accumulation in the inflammatory region due to inhibition of their attachment to the capillary vessels, IL-8 secretion from the epithelial cells and secretion of IL-8 and LTB₄ from the neutrophil, itself. Its beneficial effects in diffuse panbronchiolitis also include a reduction of superoxide production, and reduction of the proteolytic enzyme levels in lungs.

Azithromycin has been shown to significantly improve lung function, but the underlying mechanism was unclear (Jaffe et al., Lancet (1998), 351: 420), while roxithromycin was reported to suppress the growth of nasal polyp fibroblasts (Nonaka et al., Am. J. Rhinol. (1999), 13: 267-272, Yamada et al., Am. J. Rhinol. (2000), 14: 143-148).

While strong evidence in published literature exists that macrolides with a 14-membered ring such as erythromycin, clarithromycin and roxithromycin inhibit in vitro IL-8 production and neutrophil chemotaxis, evidence even in vitro is limited that macrolides with a 15-membered ring such as azithromycin exert a similar anti-inflammatory action (Criqui et al., Eur. Respir. J. (2000), 15: 856-862).

In U.S. Pat. No. 4,886,792 inhibitory effects on neutrophil degranulation of 15-membered macrolactones were described, but these lacked the sugar substituents of azithromycin. Azithromycin has been reported to induce apoptosis in human neutrophils in vitro, but was without effect on oxidative metabolism or IL-8 production (Koch et al., J. Antimicrob. Chemother. (2000), 46: 19-26). Only one study has shown azithromycin to inhibit neutrophil chemotaxis and active oxygen generation in vitro (Sugihara, Kansenshogaku Zasshi J. Jpn. Assoc. Infec. Dis. (1997), 71: 329-336). Also, azithromycin has been shown not to change TNFα, IL-1β or IL-6 levels of alveolar macrophages or blood (Aubert et al., Pul. Pharmacol. Ther. (1998), 11: 263-269).

The possibility that azithromycin, by virtue of its 15-membered ring, lacks the requisite structure conferring anti-inflammatory activity to the 14-membered macrolides has been suggested and is made more likely by the observation that 16-membered macrolides such as josamycin do not reduce IL-8 production (Takizawa et al., Am. J. Resp. Crit.Care Med. (1997), 156: 266-271; Criqui et al., Eur. Respir. J. (2000), 15: 856-862).

In comparison with macrolide antibiotics having a 14-membered ring macrolide compounds with a 15-membered ring possess several advantages. For example erythromycin whose structure is characterised by a 14-membered aglucone ring is in acidic medium easily converted into anhydroerythromycin, which is an inactive C-6/C-12 metabolite of a spiroketal structure (Kurath et al., Experienta (1971), 27: 362). In contrast to its parent antibiotic erythromycin azithromycin exhibits an improved stability in acidic medium. Furthermore, azithromycin exhibits a significantly higher concentration in tissues. Due to its improved in vitro activity against gram-negative microorganisms there was even tested the possibility of a one-day dose (Ratshema et al., Antimicrob. Agents Chemother. (1987), 31: 1939).

Thus, the technical problem underlying the present invention is to provide improved means, in particular improved processes and applications useful for the therapy of neutrophil-dominated non-infective inflammatory diseases, in which the active ingredient exhibits the advantageous anti-inflammatory activities of macrolide compounds having a 14-membered lactone ring as well as the improved stability and high tissue concentration of macrolide compounds having a 15-membered ring.

The present invention solves the above problem by the use of an active ingredient selected from the group consisting of azithromycin, a pharmaceutically acceptable derivate thereof, a pharmaceutically acceptable hydrate thereof, a pharmaceutically acceptable complex or chelate thereof and a pharmaceutically acceptable salt thereof, for the production of pharmaceutical compositions for the treatment of neutrophil-dominated, non-infective inflammatory diseases in human beings and animals.

In contrast to the limited effects of azithromycin on neutrophil function in vitro described in the art according to the present invention it has been surprisingly found that azithromycin administered to humans in vivo has a broad range of anti-inflammatory activities and is highly useful in the therapy of inflammatory diseases characterized by neutrophil infiltration and neutrophil associated tissue damage.

In a trial conducted on healthy volunteers the influence of azithromycin on selected inflammation-relevant parameters was followed up. Thereby it was found that the administration of azithromycin stimulates the degranulation of human neutrophils as shown by a strong change of the concentration of primary azurophilic granular enzymes, such as myeloperoxidase (MPO), N-acetyl-β-D-glucosaminidase (NAGA) and β-glucuronidase.

The biological relevance of MPO activity in granulocytes is a strong oxygen-dependent antimicrobial activity connected to mobilisation of all granules in the inflammatory granulocytes in the inflammation process, especially after phagocytic stimulus by immune complexes. After azithromycin application MPO activities in blood smear neutrophils strongly decreased and returned to baseline only after 28 days. Thereby it was found that degranulation presented with lower MPO neutrophil density as determined with cytochemistry was associated with lower MPO ELISA concentrations in neutrophil lysates.

N-acetyl-β-D-glucosaminidase (NAGA) and β-glucuronidase are lyosomal enzymes, both of which are located in azurophilic (primary or peroxidase-positive) granules of neutrophils. Since during inflammation degranulation of neutrophils occurs, both enzymes are markers of degranulation and can be used for estimation of neutrophil reactivity. The studies on azithromycin showed that after azithromycin application the activity of NAGA in serum increased considerably. Even 28 days after the last azithromycin dose serum NAGA values were still 70% higher than initial values. The increase in NAGA in serum was accompanied by a decrease in enzyme activity in PMN. The activity of β-glucuronidase in serum did not show any changes during the first day after the last azithromycin dose but afterwards increased. 28 days after the last dose of azithromycin the activity of β-glucuronidase was 40% higher than initially. Activities of β-glucuronidase in PMN decreased within the next hours after the last azithromycin dose but than increased. 28 days after the last azithromycin dose β-glucuronidase activity in PMN was much higher than initially.

Furthermore, according to the invention it was shown that azithromycin inhibits the generation of reactive oxygen species from stimulated neutrophils as demonstrated by the inhibition of chemiluminescence generated from stimulated neutrophils. That azithromycin is an inhibitor of neutrophil oxidative burst was further demonstrated by using a cytochrom c assay system. The studies also revealed that azithromycin has also a long-term effect on the concentration of cellular gluthatione peroxidase (GSHPx) and gluthatione reductase, two enzymes that control the biological effects of free radicals which have been implicated in the pathogenesis of a large number of diseases. Free radical production and disturbance in redox status can modulate the expression of a variety of inflammatory molecules, affecting certain cellular processes leading to inflammatory processes. Thus azithromycin provides a basis for the treatment of a variety of diseases such as COPD in which neutrophil radical production becomes excessive.

The studies also confirmed that azithromycin induces apoptosis, i.e. the programmed cell death, of certain cell types. Apoptosis is an important mechanism to complete an immune response. A three-day administration of azithromycin exerted a delayed pro-apoptotic effect on granulocytes, as indicated by the morphology of blood smear. The number of apoptotic cells reached its maximum 28 days after the last azithromycin dose suggesting a decreased number of active, potentially damaging neutrophils.

In the study other anti-inflammatory effects of azithromycin were also detected. In contrast to previous studies (Koch et al., J. Antimicrob. Chemother. (2000), 46: 19-26) it was found according to the invention that azithromycin has a marked inhibitory effect on the release of IL-8 and also GRO-α. Interleukin-8 (IL-8) is a member of the neutrophil-specific CXC subfamiliy of chemokines. It is a potent neutrophil chemotactic and activating factor (Oppenheim, Ann. Rev. Immunol. (1999), 9: 617). IL-8 is expressed in response to inflammatory stimuli. IL-8 delays spontaneous and TNF-α-mediated apoptosis of human neutrophils. In contrast to the effect on IL-8, azithromycin increases gradually the serum concentration of the cytokine IL-1, whereby the highest IL-1 concentration was found 24 h after the last azithromycin dose. However, the serum concentration of another cytokine, IL-6, was continuously decreased.

In contrast to earlier reports (Semaan et al., J. Cardiovasc. Pharmacol. (2000), 36: 533-537) in which azithromycin treatment did not significantly affect the plasma levels of soluble VCAM, studies conducted according to the present invention clearly showed a marked decrease of plasma levels of sVCAM already 24 h after azithromycin treatment.

The results obtained according to the invention demonstrate that a three-day treatment of healthy human subjects, with a standard antibacterial dosage regimen of azithromycin, exerts acute effects on neutrophil granular enzymes, oxidative burst, oxidative protective mechanisms and neutrophil chemokines and circulating IL-1, IL-6, IL-8, as well as delayed effects on neutrophil apoptosis and soluble adhesion molecules.

According to the present invention, therefore, azithromycin can be used as a valuable prophylactic and/or therapeutic agent in neutrophil-dominated, non-infective inflammatory diseases.

The following definitions are set forth to illustrate and define the meaning and scope of the various terms used to describe the present invention.

The term “neutrophil-dominated non-infective inflammatory disease” refers to inflammatory diseases, disorders or conditions which result from tissue damage, chemical irradiation or immune processes, but not from the invasion of microorganisms such as viruses, bacteria, fungi, protozoa or the like, und which are characterised by infiltration of the inflamed tissue by neutrophils which are the first inflammatory cells to enter the tissue and to amplify the inflammatory response. In some of non-infective inflammatory diseases neutrophils remain the dominant cell type within the inflamed area, even when the response is prolonged because of the continued presence of stimuli for neutrophil infiltration and activation. Examples therefore are chronic obstructive pulmonary disease (COPD), adult respiratory distress syndrome (ARDS) and neutrophilic dermatoses. Other neutrophil-dominated non-infective inflammatory diseases include diseases which have an underlying stimulus to the chronicity of the pathology, which is not dependent on neutrophils. For example autoimmune diseases are mainly due to the development of immune responses to normal structural components of the body and involve activation of T lymphocytes, with the possible production of autoantibodies by B lymphocytes. In rheumatoid arthritis (RA), for example, immune reactions are directed against structural components of the joints. However, in RA and other autoimmune diseases acute flare-ups occur, which are characterised by intense neutrophil infiltration and activation. These active phases of chronic autoimmune inflammation are neutrophil-dominated, for instance resulting in pronounced accumulation of neutrophils in the synovial fluids of patients with RA. In some autoimmune diseases, the generation of autoantibodies is pronounced, leading to deposition in the tissue of immune complexes of antigen and autoantibody and activation of the complement system. Neutrophils enter the tissue in an attempt to engulf the immune complexes and the neutrophil infiltration and activation is exacerbated by activated complement factors. An example of this type of disease is a renal disease, in particular glomerulonephritis resulting in pronounced kidney damage.

Therefore, the term “neutrophil-dominated non-infective inflammatory disease” includes, without being restricted to, chronic obstructive pulmonary disease (COPD), adult respiratory distress symptome (ARDS), bronchitis, bronchiectasis, emphysema, cystic fibrosis, inflammatory bowel disease, gouty arthritis, autoimmune diseases characterised by acute neutrophil-dominated phases, such as rheumatoid arthritis, autoimmune diseases, in which neutrophil infiltration is exacerbated by activated complement factors, such as glomerulonephritis, and skin diseases, in particular all kinds of neutrophilic dermatoses including psoriasisform dermatoses, such as psoriasis and Reiter's syndrome, autoimmune bullous dermatoses, vessel-based neutrophilic dermatoses such as leukocytoclastic vasculitis, Sweet's syndrome, pustular vasculitis, erythema nodosum and familial Mediterranean fever, and pyoderma gangrenosum.

The term “neutrophil-dominated non-infective inflammatory disease” includes also all accompanying diseases, disorders or conditions which occur as a result of a neutrophil-dominated non-infective inflammatory disease and which can affect tissues or organs of the body other than that affected by the inflammatory disease itself. An example therefore are extraintestinal diseases such as uveitis and chronic hepatitis which can result from inflammatory bowel disease.

The term “active ingredient” or “active agent” refers to any substances which can affect or recognise biological cells or parts thereof, in particular cell organelles or cellular components. Such active ingredients or agents are of a chemical nature. In particular, such active ingredients or agents are diagnostics or therapeutics. In the context of the present invention the term “active ingredients” or “active agents” refers in particular to therapeutics, i.e. substances, which can be administered as a preventive measure or during the course of a disease, disorder or condition to organisms in need of such a treatment in order to prevent or to reduce or to abolish a disease, disorder or condition, in particular a neutrophil-dominated non-infective inflammatory disease.

In the context of the present invention, the term “treatment” refers to a prophylactic and/or therapeutic effect of a drug or medicament which in turn is defined as a pharmaceutical composition comprising a pharmaceutically or diagnostically effective compound in combination with at least one additive, such as a carrier.

“Azithromycin” refers to the macrolide compound N-methyl-11-aza-10-deoxo-10-dihydroerythromycin A (9-deoxo-9-dihydro-9a-methyl-9a-aza-9a-homoerythromycin A) with a 15-membered azalactone ring which can be obtained by the Beckmann rearrangement of erythromycin A-oxime followed by Eschweiler-Clarke reductive N-methylation essentially as described in U.S. Pat. No. 4,517,359, U.S. Pat. No. 4,328,334 and BE 892,357, whereby the disclosure contents of these documents with regard to the methods for production of azithromycin are completely incorporated in the disclosure content of the present application.

The term “pharmaceutically acceptable derivative thereof” refers to non-toxic functional equivalents or derivatives of azithromycin, which can be obtained by substitution of atoms or molecular groups or bonds of the azithromycin molecule, whereby the basic structure of azithromycin is not changed, and which differ from the azithromycin structure in at least one position. The term “pharmaceutically acceptable derivative” includes for example O-methyl derivatives of azithromycin which can be obtained essentially as described in U.S. Pat. No. 5,250,518, whereby the disclosure content of this document with regard to the methods for production of O-methyl derivatives is completely incorporated in the disclosure content of the present application.

The term “pharmaceutically acceptable derivative” includes also esters of azithromycin which retain, upon hydrolysis of the ester bond, the biological effectiveness and properties of azithromycin and are not biologically or otherwise undesirable. Techniques for the preparation of pharmaceutically acceptable esters are for instance disclosed in March Advanced Organic Chemistry, 3rd Ed., John Wiley & Sons, New York (1985) p. 1152. Pharmaceutically acceptable esters useful as prodrugs are disclosed in Bundgaard, H., ed., (1985) Design of Prodrugs, Elsevier Science Publishers, Amsterdam.

The term “pharmaceutically acceptable hydrate thereof” refers to non-toxic solid or fluid compounds of azithromycin retaining the biological activities of azithromaycin and generated by the process of hydration whereby one or more molecules of water associate with the azithromycin molecule due to dipole forces. The term includes for example mono- and dihydrates of azithromycin.

The term “pharmaceutically acceptable salts” refers to the non-toxic alkali metal, alkaline earth metal, and ammonium salts commonly used including the ammonium, barium, calcium, lithium, magnesium, potassium, protamine zinc salts and sodium, which are prepared by methods known in the art. The term also includes non-toxic; i.e. pharmaceutically acceptable acid addition salts, which are generally prepared by reacting azithromycin with a suitable organic or inorganic acid, such as acetate, benzoate, bisulfate, borate, citrate, fumarate, hydrobromide, hydrochloride, lactate, laurate, maleate, napsylate, oleate, oxalate, phosphate, succinate, sulfate, tartrate, tosylate, valerate, etc.

The term “pharmaceutically acceptable acid addition salt” refers to salts which retain the biological effectiveness and properties of the free bases and which are not biologically or otherwise undesirable, formed with inorganic acids such as hydrobromic acid, hydrochloric acid, nitric acid, phosphoric acid, sulfuric acid, and organic acids such as acetic acid, benzoic acid, cinnamic acid, citric acid, ethanesulfonic acid, fumaric acid, glycolic acid, maleic acid, malic acid, malonic acid, mandelic acid, menthanesulfonic acid, oxalic propionic acid, p-toluenesulfonic acid, pyruvic acid, salicylic acid, succinic acid, tartaric acid, etc.

The salts of the invention can be obtained by dissolving azithromycin in an aqueous or aqueous/alcoholic solvent or in other suitable solvents with an appropriate base and then isolating the obtained salt of the invention by evaporating the solution, by freezing and lyophilization or by addition of another solvent, e.g. diethylether, to the aqueous and/or alcoholic solution of the azithromycin salt including the separation of unsoluble crude salt. For the preparation of alkali azithromycin salts, alkali metal carbonates or hydrogencarbonates are preferably used. The prepared salts are freely soluble in water.

The term “pharmaceutically acceptable complex or chelate thereof” refers to non-toxic complexes and chelates of azithromycin with bivalent and/or trivalent metals which can be obtained essentially as described in U.S. Pat. No. 5,498,699, whereby the disclosure content of this document with regard to the methods for production of complexes and chelates of azithromycin is completely incorporated in the disclosure content of the present application. As complex- and chelate-forming metals, metals of the II and III group which can form physiologically tolerated compounds, in particular Mg²⁺, Al³⁺, Fe³⁺, Rh³⁺, La³⁺ and Bi³⁺ can be used. Preferably the ratio of azithromycin to metal is in the range of 1:1 to 1:4. In order to obtain complexes and chelates of azithromycin the antibiotic is reacted in form of a free base or salt, in particular as a hydrochloride, with a salt of a bivalent and/or trivalent metal in a ratio of 2:1 at ambient temperature in an aqueous solution or in a mixture of water/alcohol at a pH of 8.0 to 11.0 with a metal hydroxide and/or carbonate, subsalicylate or a gel thereof. Preferred examples include chelates of azithromycin with antacids chosen from the group of salts of Al, Mg and Bi, chelates of azithromycin with sucralfate and chelates of azithromycin with bismuth-subsalicylate which are in the form of a gel.

The term “pharmaceutically or therapeutically acceptable carrier” refers to a carrier medium which does not interfere with the effectiveness of the biological activity of the active ingredients and which is not toxic to the host or patient.

The active ingredient selected from the group consisting of azithromycin, a pharmaceutically acceptable derivate thereof, a pharmaceutically acceptable hydrate thereof, a pharmaceutically acceptable complex or chelate thereof and a pharmaceutically acceptable salt can also be administered to animals, including mammals such as rodents and primates, including humans, to prevent or to reduce or to abolish neutrophil-dominated non-infective inflammatory diseases. Thus, the present invention encompasses methods for therapeutic treatment of such disorders or diseases that comprise administering an active ingredient of the invention in amounts sufficient to reach the desired effect of azithromycin in vivo. For example, the active agent or ingredient of the present invention can be administered in a therapeutically or pharmaceutically effective amount to treat a variety of non-infective inflammatory diseases, including but not limited to COPD, ARDS and neutrophilic dermatoses.

“Therapeutically or pharmaceutically effective amount” as applied to azithromycin or the azithromycin containing compounds and compositions of the present invention refers to the amount of a compound or composition sufficient to induce a desired biological result. That result can be alleviation of the signs, symptoms, or causes of a disease, or any other desired alteration of a biological system. In the present invention, the result will for instance in a particularly preferred embodiment involve preventing, abolishing and/or reducing the symptoms or causes of a neutrophil-dominated non-infective inflammatory condition by acute effects on neutrophil granular enzymes, oxidative burst, oxidative protective mechanisms and neutrophil chemokines and circulating IL-1, IL-6, IL-8, as well as delayed effects on neutrophil apoptosis and soluble adhesion molecules. In a preferred embodiment the active ingredients of the present invention will be administered prophylactically prior to the outbreak of a neutrophil-dominated non-infective inflammatory disease.

Accordingly, the present invention also provides pharmaceutical compositions comprising, as an active ingredient azithromycin, a pharmaceutically acceptable derivate thereof, a pharmaceutically acceptable hydrate thereof, a pharmaceutically acceptable complex or chelate thereof and a pharmaceutically acceptable salt in association with a pharmaceutical carrier or diluent. The compositions of this invention can be administered systematically or topically, in particular by intravascular oral, pulmonary, parenteral, e.g. intramuscular, intraperitoneal, intravenous (IV) or subcutaneous injection or inhalation, e.g. via a fine powder formulation, transdermal, nasal, vaginal, rectal, or sublingual routes of administration and can be formulated in dosage forms appropriate for each route of administration. The active agent or ingredient is adminstered preferably in a pharmaceutically effective amount.

Solid dosage forms for oral administration include capsules, lingualettes, tablets, pills, powders, liposomes, patches, time delayed coatings and granules. In such solid dosage forms, the active compound is admixed with at least one inert pharmaceutically acceptable carrier such as lactose, sucrose, or starch. Such dosage forms can also comprise additional substances other than inert diluents, e.g., lubricating agents such as magnesium stearate. In the case of capsules, tablets, and pills, the dosage forms may also comprise bulking and/or buffering as well as flavouring agents. Tablets and pills can additionally be prepared with enteric coatings.

Liquid dosage forms for oral administration include pharmaceutically acceptable emulsions, solutions, suspensions, syrups, with the elixirs containing inert diluents commonly used in the art, such as water. Besides such inert diluents, compositions can also include adjuvants, such as salts for varying the osmotic pressure, pH-adjusting compounds, skin penetration agents, wetting agents, emulsifying and suspending agents, and sweetening, flavouring, and perfuming agents.

Pharmaceutical compositions according to the present invention for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatine, and injectable organic esters such as ethyl oleate. Such dosage forms may also contain adjuvants such as preserving, wetting, emulsifying, and dispersing agents. They may be sterilised by, for example, filtration through a bacteria retaining filter, by incorporating sterilising agents into the compositions, by irradiating the compositions, or by heating the compositions. They can also be manufactured using sterile water, or some other sterile injectable medium, immediately before use.

Formulations for injection will comprise a physiologically-acceptable medium, such as water, saline, PBS, aqueous ethanol, aqueous ethylene glycols and the like. Water soluble preservatives which may be employed include sodium bisulfite, sodium thiosulfate, ascorbate, benzalkonium chloride, chlorobutanol, thimerosal, phenylmercuric borate, parabens, benzyl alcohol and phenylethanol. These agents may be present in individual amounts of from about 0.001 to about 5% by weight and preferably about 0.01 to about 2%. Suitable water soluble buffering agents that may be employed are alkali or alkaline earth carbonates, phosphates, bicarbonates, citrates, borates, acetates, succinates and the like, such as sodium phosphate, citrate, borate, acetate, bicarbonate and carbonate. Additives such as carbomethylcellulose may be used as a carrier in amounts of from about 0.01 to about 5% by weight. The formulation will vary depending upon the purpose of the formulation, the particular mode employed for treating a disease, the intended treatment, etc.

Compositions for rectal or vaginal administration are preferably suppositories which may contain, in addition to the active substance, excipients such as cocoa butter or a suppository wax. Compositions for nasal or sublingual administration are also prepared with standard excipients well known in the art.

The compositions containing the active agent or ingredient of the present invention can be administered for prophylactic and/or therapeutic treatments. In therapeutic applications, compositions are administered to a patient already suffering from a disease, as described above, in an amount sufficient to cure or at least partially arrest the symptoms of the disease and its complications, i.e. a therapeutically effective amount.

In prophylactic applications, compositions containing the active agent or ingredient of the present invention are administered to a patient susceptible to or otherwise at risk of a particular disease. Such an amount is defined to be a “prophylactically effective dose.” In this use, the precise amounts again depend upon the patient's state of health and weight.

The pharmaceutical compositions of the present invention may also be administered in the form of a depot, such as a slow release composition. Such a slow release composition may include particles of the active agent or ingredient in a matrix, made e.g. from collagen.

The quantities of the active agent or ingredient necessary for effective therapy will depend upon many different factors, including means of administration, target site, physiological state of the patient, and other medicants administered.

The active agent or ingredient of the present invention selected from the group consisting of azithromycin, a pharmaceutically acceptable derivate thereof, a pharmaceutically acceptable hydrate thereof, a pharmaceutically acceptable complex or chelate thereof and a pharmaceutically acceptable salt thereof are effective in treating neutrophil-dominated non-infective inflammatory diseases when administered in a range of from about 10 mg to about 2000 mg per day, in particular from about 30 to about 1500 mg. The specific dose employed is regulated by the particular condition being treated, the route of administration, as well as by the judgement of the attending clinician depending upon factors such as the severity of the condition, and the age and general condition of the patient.

The active agents or ingredients of the present invention may be administered alone or together with other medicaments currently used for the treatment of neutrophil-dominated non-infective inflammatory diseases such as non-steroidal anti-inflammatory agents, such as methyl xanthine non-steroidal anti-inflammatory agents, steroidal anti-inflammatory agents, immunomodulating agents, immunosuppressive agents, bronchodilating agents, antirheumatic agents, corticosteroids, β2-agonists, cholinergic antagonists, and the like, whereby the dose of the latter can possibly be reduced by 50% or 25% due to the anti-inflammatory effects of the active ingredients of the present invention.

The composition, preferably the water-soluble composition, of the invention may further contain a water-soluble protein injectable into body fluids without showing any substantial pharmacological activity at the concentration used in one unit dosage form of the present invention (hereinafter, “water-soluble protein”). As such a water-soluble protein, serum albumin, globulin, collagen and/or gelatine are preferred. This protein can be added in an amount generally employed in injectable pharmaceutical compositions. Thus, for example, the weight ratio between the water-soluble protein and the active agent or ingredient of the present invention is about 0.0001:1 to 100:1, preferably about 0.001:1 to about 10:1 or more preferably about 0.01:1 to about 1:1.

Continuing, the invention also relates to the aforementioned active agents or ingredients themselves and compositions containing them, in particular, in dried and/or pure form or in an aqueous or aqueous/alcoholic solution. The pH of a solution prepared from the water-soluble composition or an active agent of the present invention should be such that said pH will not exert any adverse influence upon the activity of the pharmacologically active peptide, but is within an acceptable range for injections in general and further, such that said pH will neither cause a great change in viscosity of the solution nor allow formation of a precipitate or the like. Thus the solution should preferably have a pH of about 4 to 7, preferably 5 to 6, in particular 5.3 to 5.5.

When the water-soluble composition of the invention is converted into an aqueous solution for administration, the concentration of the pharmacologically active agent or ingredient or salt thereof in said solution should preferably be about 0.0000001 to 10% (w/v), more preferably about 0.000001 to 5% (w/v) or most preferably about 0.00001 to 1% (w/v).

The composition of the present invention should preferably have a unit dosage form containing the pharmacologically active agent or ingredient of the invention and, if necessary, together with further additives such as the above mentioned water-soluble protein. Thus, for example, the two or three components mentioned above are made to occur in an ampule or vial by dissolving or suspending them in sterile water or sterile physiological saline. In this case, the method of preparation may comprise admixing a solution of the pharmacologically active agent or ingredient and further, if necessary, a solution of the additive or adding the additive in a powder form to a solution of the pharmacologically active agent or ingredient or any other combination of adequate procedures. The dosage form may also be prepared by adding sterile water or sterile physiological saline to a lyophilizate or vacuum-dried powder in which the pharmacologically active agent, and if necessary the additive, coexist. This unit dosage form may contain one or more conventional additives such as pH adjusting agents (e.g. glycine, hydrochloric acid, sodium hydroxide), local anesthetics (e.g. xylocaine hydrochloride, chlorobutanol), isotonizing agents (e.g. sodium chloride, mannitol, sorbitol), emulsifiers, adsorption inhibitors (e.g. Tween® 60 or 80), talcum, starch, lactose and tragacanth, magnesium stearate, glycerol, propylen glycol, preserving agents, benzyl alcohol, methylhydroxy benzoate and/or oleum arachid hydrogen. This unit dosage form may further contain pharmaceutically acceptable excipients such as polyethylene glycol 400 or dextran.

The composition of the present invention is made by admixing these ingredients according to a conventional method. The goal of admixing the ingredients of the present composition should be such that the activity of the pharmacologically active agent is maintained and bubble formation minimised during the process. The ingredients are put into a vessel (for example a bottle or drum) either at the same time or in any order. The atmosphere in the vessel can be, for example, sterile clean air or sterile clean nitrogen gas. The resultant solution can be transferred to small vials or ampules and can be further subjected to lyophilization.

The liquid form or the lyophilizate powder form of the composition of the present invention may be dissolved or dispersed in a solution of a biodegradable polymer such as poly(lactic-glycolic) acid copolymer, poly(hydroxybutyric acid), poly(hydroxybutyric-glycolic) acid copolymer, or the mixture of these, and then may be formulated, for example, to films, microcapsules (microspheres), or nanocapsules (nanospheres), particularly in the form of soft or hard capsules.

In addition, the composition of the present invention encapsulated in liposomes comprising phospholipids, cholesterol or the derivatives of these can be further dispersed in physiological saline or a hyaluronic acid solution dissolved in physiological saline.

The soft capsule may be filled with the liquid form of the composition of the present invention. The hard capsule may be filled with the lyophilizate powder of the composition of the present invention, or the lyophilizate powder of the present composition may be compressed to tablets for rectal administration or oral administration respectively.

Of course, the composition of the present invention can be supplied in a pre-filled syringe for self-administration.

Although only preferred embodiments of the invention are specifically described above, it will be appreciated that modifications and variations of the invention are possible without departing from the spirit and intended scope of the invention. Further preferred embodiments of the present invention are listed in the claims.

### EXAMPLE

A trial on healthy volunteers was conducted and the influence of azithromycin given in a dosage of 3×500 mg on selected inflammation-relevant parameters was followed up.

Drug Administration, Blood Sampling and Plasma

Each subject received two standard 250 mg capsules of azithromycin (Sumamed®, PLIVA Zagreb) on three consecutive days. Immediately before the treatment and 2 h and 30 min, 24 h and 28 days after the third and last dose of azithromycin blood was collected from the cubital vein into EDTA-containing tubes. Aliquots were taken for cell counting, smear preparation, polymorphonuclear cell and serum isolation.

Analysis of Primary Azurophilic Granular Enzymes

Leucocyte granules are membrane-bound organells containing an array of antimicrobial proteins. Apart from containing degradative enzymes that may be extracellulary secreted from the neutrophil or else discharged into phagocytic vesicles, the membranes of many types of these granules and vesicles contain important molecules such as certain receptors (e.g. fMLP receptor) and cytochrome b of NADPH oxidase.

a) Analysis of Myeloperoxidase

The enzyme myeloperoxidase (MPO) is a 135,000 dalton protein containing two heavy and two light chains of 55,000 and 15,000 daltons. MPO is situated in primary or azurophil granules of granulocytic cells. The function of MPO is to provide reactive oxygen metabolites that are essential for microbicidal activity of neutrophils. The generation of oxygen metabolites is dependent on components of MPO-negative granules (which harbour the flavocytochrome b₅₅₈, an essential component of the NADPH oxidase) and on components of azurophil MPO positive granules. MPO transforms the relatively innocuous product of the NADPH oxidase, H₂0₂, to hypochlorous acid. The biological relevance of MPO activity in granulocytes is a strong oxygen-dependent antimicrobial activity connected to mobilisation of all granules in the inflammatory granulocytes in the inflammation process, especially after phagocytic stimulus by immune complexes.

The activity of MPO was assessed from the intensity of staining of neutrophils in blood smears and in cell lysates by ELISA. After fixation in ethanol-formaldehyde, smears were incubated in a substrate solution containing hydrogen peroxide and benzidine (SIGMA). After incubation, smears were counterstained with Giemsa solution. MPO value positivity_of 100 granulocytes was evaluated and scored from 0 to 4+ on the basis of the intensity of the precipitated dye in cytoplasm. Therefore, the value of the score could be from 0 to 400. Normal values of score range (290-390) were taken from this study before azithromycin administration. MPO activity was also evaluated on the digital image of smear taken with a digital camera under the high magnification (×1000) of light microscope. MPO activities in blood smear neutrophils decreased from 2 h and 30 min to 24 h after the last azithromycin dose and returned to baseline after 28 days (Table 1). The concentration of MPO enzyme protein determined by ELISA in lysates of neutrophils is shown in Table 1. The change in neutrophil enzyme protein followed the same pattern as that in intracellular enzyme activity, decreasing from 2 h and 30 min to 24 h after the last dose of azithromycin and returning to baseline after 28 days. Both methodological approaches of MPO determination confirmed each other. Degranulation presented with lower MPO neutrophil density determined_with cytochemistry was associated with lower MPO ELISA concentrations in neutrophil lysates.

b) Analysis of N-acetyl-β-D-glucosaminidase (NAGA) and β-glucuronidase

Glycosidases are enzymes that catalyse hydrolysis of glycosidic bonds of oligosacharides and other glycosides. They are specific to the glycosidic part of substrate molecule. N-acetyl-β-D-glucosaminidase (NAGA) and β-glucuronidase are such enzymes. They are lysosomal enzymes, both located in azurophilic (primary; peroxidase-positive) granules of neutrophils. Since degranulation of neutrophils is present during inflammation, many authors choose these enzymes as markers of degranulation and for estimation of neutrophil reactivity. The catalytic concentration of both enzymes in serum and in neutrophil lysates was determined using the fluorimetric method described by O'Brien et al. (New Engl. J. Med. (1970) 283: 15-20) for NAGA and Glaser & Sly (J. Lab. Clin. Med. (1973) 82: 969) for β-glucuronidase.

The results showed (Table 1) that activity of NAGA in serum increased about 30% 2 h and 30 min after the last dose. 24 hours after the last dose it was approximately 50% higher than the initial values. 28 days later, serum NAGA values were still 70% higher than initial values. The increase in NAGA in serum was accompanied by a decrease in enzyme activity in PMN. 2 h and 30 min after the last dose a decrease of about 70% in NAGA in granulocytes was determined. 24 hours later, NAGA activity in PMN increased by about 30% but it was still about 40% lower compared to initial values. After 28 days the activity of NAGA increased 40% over the initial values (Table 1).

The activity of β-glucuronidase in serum did not show any changes during the first 24 hours after the last dose. 28 days later serum values were about 40% higher than initially. Activities of β-glucuronidase in PMN decreased by about 20% after 2 h and 30 min and by about 50% 24 hours later compared to initial values. However 28 days later, β-glucuronidase activity in PMN was much higher (about 300%) compared to the initial values (Table 1).

When analysing activities of glycosidases it is obvious that azithromycin in healthy volunteers induced release of 40-50% enzymes from azurophilic granules within 24 hours after the last dose. The decrease of NAGA activity in PMNs was accompanied by an increase in serum. Serum activities of the two enzymes showed a slight increase over baseline (before azithromycin) 2 h and 30 min and 24 h after the last dose of the drug, increasing a further 28 days later (Table 1).

In contrast, activities of the two enzymes in neutrophil lysates decreased in the hours after the last dose of azithromycin, the fall in NAGA activity being maximal after 2 h and 30 min and returning to baseline after 28 days. The cellular activity of β-glucuronidase was still falling 24 h after the last dose of azithromycin and increased to well above baseline levels after 28 days (Table 1).

In summary, enzymes released from neutrophil primary azurophilic granules tended to be present in serum at slightly higher activities 2 h and 30 min to 24 h after azithromycin administration, while over the same time period, their activities were lower in peripheral blood neutrophils, suggesting that they were being released by degranulation. NAGA was released early after azithromycin, while MPO and β-glucuronidase exhibited a delayed release. Recovery of these enzyme activities also varied.

Studies on Neutrophil Oxidative Burst

All aerobic organisms use oxygen for the production of energy. However, there are many indications that the advantages of using oxygen are associated with a risk that the oxidative process may also cause injury. During phagocytosis when neutrophils are stimulated, they undergo an oxidative burst, with generation and release of reactive oxygen metabolites. These reactive oxygen species serve as the major mechanism by which phagocytes mediate their antimicrobial effect. The reactions are characterised by rapid oxygen uptake followed by reduction of oxygen to superoxide (O₂⁻). This is catalysed by NADPH oxidase using NADPH or NADH as electron donor. When these defence mechanisms are directed inappropriately, tissue damage occurs.

a) Determination of Chemiluminescence Generation

The generation of reactive oxygen species by activated cells is frequently determined by the measurement of chemiluminescence (CL). The radical species formed react with a photon-producing chemical (e.g. Luminol) and the resulting light emission is measured with a photocell. Chemiluminescence is detectable as a result of the stimulation (e.g. fMLP) of leucocytes and is a measure of their oxidative cytotoxic activity (Allen et al., Biochem. Biophys. Res. Commun. (1972), 47: 679).

The results of the study presented in Table 1 show, that azithromycin inhibits chemiluminescence generated from stimulated neutrophils isolated from the blood of humans treated with azithromycin.

b) Cytochrome C Assay System

Neutrophils were incubated with cytochrome c and stimulated with fMLP (Cohen and Chovaniec, 1978, J. Clin. Invest. 61: 1081-1087). Absorbances at 550 nm and 540 nm were recorded and the results were expressed as delta A.

The oxidative burst of neutrophils in response to the bacterial peptide fMLP was inhibited by the 3 day dosing with azithromycin (Table 1). Using both cytochrome c and luminol as assay systems, inhibition was already detectable 2 h and 30 min after the last dose of azithromycin, was greater after 24 h and had not returned to normal 28 days later.

Consequently, azithromycin is to be considered as an inhibitor of the oxidative burst. Thus, azithromycin provides a basis for a variety of diseases in which neutrophil radical production (oxidative burst) becomes excessive such as COPD.

Analysis of Glutathione Peroxidase and Glutathione Reductase

Oxygen free radicals and lipid peroxides have been implicated in the pathogenesis of a large number of diseases. The biological effects of free radicals are controlled in vivo by a wide range of antioxidants such as α-tocopherol (vitamin E), ascorbic acid (vitamin C), β-carotene, reduced glutathione (GSH) and antioxidant enzymes (superoxide dismutase, SOD, glutathione peroxidase GSHPx, catalase, CAT) (Benabdeslam et al., Clin. Chem. Lab. Med. (1999), 37: 511-516; Mates et al., Blood Cells Mol. (1999), 25: 103-109). Recently, antioxidant functions have been definitively linked to anti-inflammatory and/or immunosuppressive properties (Mates et al., Blood Cells Mol. (1999), 25: 103-109). Free radical production and disturbance in redox status can modulate the expression of a variety of inflammatory molecules (Sundaresan et al., Science (1995), 270: 296-299; Kaouass et al., Endocrine (1997), 6: 187-194), affecting certain cellular processes leading to inflammatory processes, both exacerbating inflammation and effecting tissue damage (Tsai et al., FEBS Lett. (1997), 436: 411-414).

Cellular glutathione peroxidase (GSHPx) is a tetrameric protein in which each of the four identical subunits contains one atom of selenium (Se) in the form of selenocysteine at the active site (Misso et al., J. Leukoc. Biol. (1998), 63: 124-130). GSHPx plays a role in H₂0₂ detoxification and converts lipid hydroperoxides to nontoxic alcohols (Akkus et al., Clin. Chim. Acta (1996), 244: 221-227); Urban et al., Biomed & Pharmacother. (1997), 51: 388-390). In this study, in healthy volunteers treated with azithromycin alterations in the PMN intracellular GSHPx activity were determined using the commercially available kit RANSEL (Randox Laboratories). GSHPx catalyses the oxidation of glutathione by cumene hydroperoxide. In the presence of glutathione reductase and NADPH the oxidised glutathione is immediately converted to the reduced form with a concomitant oxidation of NADPH to NADP⁺. The decrease in absorbance at 340 nm is measured.

Glutathione reductase is an ubiquitous enzyme that catalyses the reduction of oxidised glutathione (GSSG) to glutathione (GSH). Glutathione reductase is essential for the glutathione redox cycle that maintains adequate levels of reduced cellular GSH. GSH serves as an antioxidant, reacting with free radicals and organic peroxides, in amino acid transport, and as a substrate for the GSHPx and glutathione S-transferases in the detoxification of organic peroxides and metabolism of xenobiotics. Glutathione reductase was determined using the BIOXYTECH® GR-340™ colorimetric assay for glutathione reductase (OXIS International, Inc.). Briefly, oxidation of NADPH to NADP⁺ is catalysed by a limiting concentration of glutathione reductase.

GSHPx activity in neutrophil lysates (expressed per number of cells) was unchanged 2 h and 30 min after the last dose of azithromycin, but decreased significantly 24 h after this last dose (Table 1). The activity had returned to baseline 28 days later. Glutathione reductase activity in cell lysates (expressed per number of cells) showed a similar tendency, decreasing significantly 2 and 30 min and 24 h after the last dose of azithromycin, returning to normal values and then reaching higher levels than normal 28 days after the treatment (Table 1).

Analysis of Apoptosis

Three-day administration of azithromycin exerted a delayed pro-apoptotic effect on granulocytes, as indicated by morphology of blood smears. The results are presented in Table 1. The number of apoptotic cells counted increased continuously after the three day dosing with azithromycin, achieving statistical significance 28 days after the last dose. An increased number of apoptotic cells suggest a decreased number of active, potentially damaging neutrophils.

Analysis of Cytokines and Chemokines

Other acute, but potentially anti-inflammatory effects of azithromycin were also detected in this study.

Interleukin-8, a member of the neutrophil-specific CXC subfamily of chemokines is a potent neutrophil chemotactic and activating factor (Oppenheim, J.J. Ann. Rev. Immunol. (1999), 9: 617). It binds to at least two G protein-coupled receptors (IL-8R1 and IL-8R2). These receptors are functionally different. Responses, such as cytososlic free Ca²⁺ changes and release of the granule enzymes, are mediated through both receptors, whereas the respiratory burst and the activation of phospholipase D depend exclusively on stimulation through IL-8R1 (Johnes et al., Proc. Natl. Acad. Sci. USA (1996), 93: 6682-6686). IL-8 is a key mediator in the recruitment of circulating neutrophils. This chemokine is expressed in response to inflammatory stimuli, and is secreted by a variety of cell types, including lymphocytes, epithelial cells, keratinocytes, fibroblasts, endothelial cells, smooth muscle cells and neutrophiles. In the latter instance, IL-8 is one of the most abundantly secreted (and most extensively) studied cytokines produced by neutrophils. Interestingly enough, neutrophils represent the primary cellular target for IL-8, to which they respond by chemotaxis, release of granule content, respiratory burst, up-regulation of cell surface receptors, increased adherence to non-stimulated endothelial cells, and transmigration across the endothelium. Agents capable of stimulating the production of IL-8 by human neutrophils are: TNF-α, IL-1β, GM-CSF, leukotriene B₄, PAF, fMLP, lactoferrin, LPs and many others (Cassatella, M. A., Adv. Immunol. (1999), 73: 369-509). IL-8 delays spontaneous and TNF-α-mediated apoptosis of human neutrophils. (Kettritz et al., Kidney Int. (1998), 53: 84-91). IL-8 is the pre-dominant C—X—C chemokine and the dominant neutrophil chemoattractant accumulating in supernatant of LPS-stimulated human alveolar macrophages (Goodmann et al., Am. J. Physiol. (1998), 275: L87-L95).

Erythromycin was reported to have an inhibitory effect on IL-8 expression in human epithelial cells and this mode of action is probably of relevance for its clinical effectiveness (Takizawa et al., Am. J. Respir. Crit. Care Med. (1997), 156: 266-271).

Roxithromycin is also capable of reducing IL-8 production in nasal polyp fibroblasts (Nonaka et al., Acta Otolaryngol. (1998) Suppl. 539: 71-75). In synoviocytes from rheumatoid arthritis, the production of IL-1α, IL-6, IL-8, GM-CSF could be inhibited by chlarithromycin (Matsuoka et al., Clin. Exp. Immunol. (1996), 104(3): 501-8). Ex vivo assessment of IL-8 production in whole blood also confirmed the potential of erythromycin for inhibiting IL-8 production (Schultz et al., J. Antimicrob. Chemother. (2000), 46: 235-240. A similar finding has recently been reported for human bronchial epithelial cells (Desaki M. et al., Biochim. Biophys Res. Commun. (2000) 267: 124-128). A recent study, however, reported a lack of azithromycin modulatory effect on IL-8 production of PMN in vitro (Koch et al., J. Antimicrob. Chemother. (2000), 46: 19-26).

Cytokine and chemokine concentrations were determined using ELISA kits. Several different response patterns were seen in serum cytokine and chemokine concentrations following three-day administration of azithromycin. Rapid and pronounced decreases in the plasma concentrations of the neutrophil-stimulating chemokine, IL-8, and GR0-α were observed 2 h and 30 min and 24 h after the last dose of azithromycin (Table 1. The concentration of IL-8 returned essentially to baseline after 28 days, while that of GRO-□ was decreased at this time.

These data clearly demonstrate the acute inhibitory effect of azithromycin on the release of IL-8 ex vivo, extending this property also to inhibition of the release of the chemokine GRO-□. It should be stated, however, that the serum chemokine concentration was measured. Therefore one cannot draw any conclusion as to the cellular source(s) of the chemokines.

The low baseline serum concentration of IL-1 gradually increased after the last dose of azithromycin, achieving statistical significance after 24 h (Table 1). The concentration had returned to baseline 28 days after azithromycin. In contrast, the serum concentration of IL-6 exhibited a continuous decrease, achieving statistical significance 28 days after the last dose of azithromycin (Table 1).

Analysis of Adhesion Molecules

In contrast to earlier reported data (Semaan et al., J. Cardiovasc. Pharmacol. (2000), 36: 533-537) in which azithromycin treatment did not significantly affect the plasma levels of soluble VCAM, in this study a decrease in serum sVCAM was observed 24 h after the last dose of azithromycin, remaining significantly reduced after 28 days, indicating that azithromycin has the potential to inhibit both the generation of neutrophil chemotactic peptides and the expression and release of adhesion molecules for activated leucocytes (Table 1). For quantitative determination of serum concentration of human sVCAM, an ELISA kit was used (R&D systems, UK)

Proteins in PMN samples were determined according to the method of Bradford (Anal. Biochem. (1976) 72: 248-254) using bovine serum albumin as a standard.

TABLE 1

| | UNITS | baseline | 2 h and 30 min | 24 hours | 28 days |
|---|---|---|---|---|---|
| DEGRANULATION | | | | | |
| myeloperoxidase (score) | | 337 ± 29 | 326 ± 26 | 315 ± 22* | 347 ± 18 |
| myeloperoxidase (density) | | 105 ± 13 | 130 ± 16* | 131 ± 17* | 115 ± 19 |
| myeloperoxidase (PMN) | μg/mg protein | 54.22 ± 12.61 | 70.85 ± 19.91 | 26.74 ± 2.51* | 70.01 ± 17.62 |
| NAGA (PMN) | nmol × 10⁻⁶ cells × min⁻¹ | 4.15 ± 01.6 | 1.13 ± 0.72* | 2.62 ± 1.6* | 5.95 ± 3.7 |
| β-glucuronidase (PMN) | nmol × 10^{-6 cells × min}⁻¹ | 4.12 ± 2.7 | 3.21 ± 2.3 | 1.58 ± 0.4* | 15.37 ± 11.4* |
| NAGA (serum) | μmol × L⁻¹ × min⁻¹ | 9.16 ± 1.6 | 11.52 ± 2.2 | 13.7 ± 1.5* | 14.87 ± 1.9* |
| β-glucuronidase (serum) | μmol × L⁻¹ × min⁻¹ | 2.88 ± 0.7 | 3.01 ± 0.6 | 2.95 ± 0.5 | 3.93 ± 1.2* |
| CYTOKINES (serum) | | | | | |
| IL-1 | pg/mL | 0.291 ± 0.11 | 0.533 ± 0.15 | 1.07 ± 0.19* | 0.29 ± 0.20 |
| IL-6 | pg/mL | 3.4 ± 1.05 | 2.7 ± 1.49 | 2.5 ± 1.48 | 1.15 ± 0.61* |
| CHEMOKINES (serum) | | | | | |
| IL-8 | pg/mL | 29.47 ± 15.44 | 10.61 ± 3.81* | 14.60 ± 10.75* | 23.03 ± 19.72 |
| GRO-α | pg/mL | 124.1 ± 33.02 | 109.6 ± 30.35* | 107.9 ± 27.83* | 90.4 ± 22.32* |
| APOPTOSIS (WBC) | apoptopic cells/1000 WBC | 0.333 ± 0.655 | 0.833 ± 1.029 | 1.417 ± 1.240 | 2.583 ± 2.02* |
| ADHESION MOLECULES | | | | | |
| sV-CAM | ng/mL | 13.59 ± 2.90 | 12.21 ± 4.12 | 10.29 ± 2.12* | 10.74 ± 2.05* |
| OXIDATIVE BURST (PMN) | | | | | |
| fMLP-luminol | A.U. | 29335 ± 1957 | 14774 ± 1175* | 5053 ± 3804* | 9879 ± 13880* |
| fMLP-cytochrom c | ΔA | 0.020 ± 0,014 | 0,007 ± 0.015* | −0,018 ± 0.010* | −0,0011 ± 0,0010* |
| GLUTATHION PEROXIDASE (PMN) | mU/10⁶PMN | 5.3 ± 2.0 | 5.3 ± 2.9 | 1.6 ± 1.3* | 8.0 ± 5.2 |
| GLUTATHION REDUCTASE (PMN) | mU/10⁶PMN | 9.63 ± 1.16 | 7.39 ± 1.23* | 7.91 ± 0.87* | 11.27 ± 2.24* |

*p < 0.01 vs baseline (Wilcoxon).

1. A method for treating neutrophil-dominated, non-infective inflammatory diseases in human beings and animals comprising administering to said human beings and animals a therapeutically or pharmaceutically effective amount of an active ingredient which comprises azithromycin, or a pharmaceutically acceptable derivative, hydrate, complex, chelate, or salt thereof.

2. The method according to claim 1, whereby the active ingredient is an 0-methyl-derivative of azithromycin.

3. The method according to claim 1, whereby the active ingredient is an ester of azithromycin.

4. The method according to claim 1, whereby the active ingredient is a monohydrate of azithromycin.

5. The method according to claim 1, whereby the active ingredient is a dihydrate of azithromycin.

6. The method according to claim 1, whereby the active ingredient is a complex or chelate of azithromycin with metal ions.

7. The method according to claim 6, whereby the ratio between azithromycin to metal is 1:1 to 1:4.

8. The method according to claim 6, whereby the metal ions are bivalent metal ions.

9. The method according to claim 6, whereby the metal ions are trivalent metal ions.

10. The method according to claim 1, whereby the active ingredient is an alkali metal, alkaline earth metal, or an ammonium salt of azithromycin.

11. The method according to claim 1, whereby the active ingredient is an acid addition salt of azithromycin.

12. The method according to claim 11, whereby the acid addition salt is formed with an inorganic acid.

13. The method according to claim 11, whereby the inorganic acid is hydrobromic acid, nitric acid, phosphoric acid or sulphuric acid.

14. The method according to claim 11, whereby the acid addition salt is formed with an organic acid.

15. The method according to claim 14, whereby the organic acid is acetic acid, benzoic acid, cinnamic acid, citric acid, ethanesulfonic acid, fumaric acid, glycolic acid, maleic acid, malic acid, malonic acid, mandelic acid, methanesulfonic acid, oxalic acid, p-toluenesulfonic acid, pyruvic acid, salicylic acid, succinic acid or tartaric acid.

16. The method according to claim 1, whereby the pharmaceutical compositions contain the active ingredient in an amount sufficient to abolish or to reduce the disease or to stop its progression.

17. The method according to claim 16, whereby the pharmaceutical compositions are administered one to three times a day in a dose of 10 mg to 200 mg active ingredient.

18. The method according to claim 17, whereby the pharmaceutical compositions are administered one to three times a day in a dose of 30 mg to 1500 mg active ingredient.

19. The method according to claim 1, whereby the pharmaceutical compositions are orally administered in solid or liquid dosage forms.

20. The method according to claim 19, whereby the solid pharmaceutical compositions for oral administration are capsules, lingualettes, tablets, pills, powders, liposomes, patches, time delayed coatings and granules.

21. The method according to claim 19, whereby the solid pharmaceutical compositions for oral administration. contain at least one inert pharmaceutically acceptable carrier.

22. The method according to claim 21, whereby the inert pharmaceutical carrier is lactose, sucrose, or starch.

23. The method according to claim 19, whereby the solid pharmaceutical compositions for oral administration comprise additional substances selected from the group consisting of lubricating agents such as magnesium stearate, bulking and/or buffering agents and flavouring agents.

24. The method according to claim 19, whereby the solid pharmaceutical compositions for oral administration are prepared with enteric coatings:

25. The method according to claim 19, whereby the liquid pharmaceutical compositions for oral administration are pharmaceutically acceptable emulsions, solutions, suspensions or syrups.

26. The method according to claim 25, whereby the liquid pharmaceutical composition for oral administration contains at least one inert pharmaceutical carrier.

27. The method according to claim 26, whereby the inert pharmaceutical carrier is water or physiological saline.

28. The method according to claim 25, whereby the liquid pharmaceutical composition for oral administration comprises additional substances, selected from the group consisting of adjuvants, salts for varying the osmotic pressure, pH-adjusting compounds, skin penetration agents, wetting agents, emulsifying and suspending agents.

29. The method according claim 1, whereby the pharmaceutical compositions are parenterally administered.

30. The method according to claim 29, whereby the pharmaceutical compositions for parenteral administration are infusions or injections.

31. The method according to claim 29, whereby the pharmaceutical compositions for parenteral administration are sterile aqueous or non-aqueous solutions, suspensions or emulsions.

32. The method according to claim 29, whereby the pharmaceutical compositions for parenteral administration comprise non-aqueous solvents or vehicles.

33. The method according to claim 32, whereby the non-aqueous solvents or vehicles are propylene glycol, polyethylene glycol, vegetable oils, such as olive oil and corn oil, gelatine, and injectable organic esters such as ethyl oleate.

34. The method according to claim 29, whereby the pharmaceutical compositions for parenteral administration comprise adjuvants such as preserving, wetting, emulsifying, and dispersing agents.

35. The method according to claim 1, whereby the pharmaceutical compositions are rectally or vaginally administered.

36. The method according to claim 35, whereby the pharmaceutical compositions for rectal or vaginal administration are suppositories, clysters or foams.

37. The method according to claim 35, whereby the pharmaceutical compositions for rectal or vaginal administration contain excipients such as cocoa butter or a suppository wax.

38. The method according to claim 1, whereby the pharmaceutical compositions for the treatment of neutrophil-dominated, non-infective inflammatory diseases contain one or more additional active ingredients useful for the treatment of such diseases selected from the group consisting of non-steroidal anti-inflammatory agents, steroidal anti-inflammatory agents, bronchodilating agents, antirheumatic agents, immunomodulating agents, immunosuppressive agents, corticosteroids, β2-agonists and cholinergic antagonists.

39. The method according to claim 38, whereby the, dose of the additional active ingredients is reduced in comparison to pharmaceutical compositions, containing exclusively one of the additional active ingredients.

40. A pharmaceutical composition for the treatment of neutrophil-dominated, non-infective inflammatory diseases in human beings and animals comprising an active ingredient which comprises azithromycin, or a pharmaceutically acceptable derivate, hydrate, complex, chelate, or salt thereof

41. The pharmaceutical composition according to claim 40, whereby the active ingredient is an O-methyl-derivative or an ester of azithromycin.

42. The pharmaceutical composition according to claim 40, whereby the active ingredient is a monohydrate or a dehydrate of azithromycin.

43. The pharmaceutical composition according to claim 40, whereby the active ingredient is a complex or chelate of azithromycin with bivalent or trivalent metal ions.

44. The pharmaceutical composition according to claim 43, whereby the ratio between azithromycin and metal ions is 1:1 to 1:4.

45. The pharmaceutical composition according to claim 40, whereby the active ingredient is an alkali metal, alkaline earth metal, or an ammonium salt of azithromycin.

46. The pharmaceutical composition according to claim 40, whereby the active ingredient is an acid addition salt of azithromycin.

47. The pharmaceutical composition according to claim 46, whereby the acid addition salt is formed with an inorganic acid, such as hydrobromic acid, nitric acid, phosphoric acid or sulphuric acid.

48. The pharmaceutical composition according to claim 46, whereby the acid addition salt is farmed with an organic acid, such as acetic acid, benzoic acid, cinnamic acid, citric acid, ethanesulfonic acid, fumaric acid, glycolic acid, maleic acid, malic acid, malonic acid, mandelic acid, methanesulfonic acid; oxalic acid, p-toluenesulfonic acid, pyruvic acid, salicylic acid, succinic acid or tartaric acid.

49. The pharmaceutical composition according to claim 40, whereby the active ingredient is contained in an amount sufficient to abolish or to reduce the disease or to stop its progression.

50. The pharmaceutical composition according to claim 40, comprising one or more additional active ingredients useful for the treatment of such diseases selected from the group consisting of non-steroidal anti-inflammatory agents, steroidal anti-inflammatory agents, bronchodilating agents, antirheumatic agents, immunomodulating agents, immunosuppressive agents, corticosteroids, β2-agonists and cholinergic antagonists.

51. The pharmaceutical composition according to claim 50, whereby the dose of the additional active ingredients is reduced in comparison to pharmaceutical compositions, containing exclusively one of the additional active ingredients.

52. A method for the production of a pharmaceutical composition comprising azithromycin, or a pharmaceutically acceptable derivative, hydrate, chelate, or salt thereof as an active ingredient, for the treatment of neutrophil-dominated, non-infective inflammatory diseases in human beings and animals which comprises
(i) admixing the active ingredient with additives and optionally with additional active ingredients useful for the treatment of such diseases,
(ii) dissolving or suspending the resulting admixture in sterile aqueous or aqueous/alcoholic solution,
(iii) adjusting the pH of the solution to a value of about 4 to 7 by the use of pH adjusting agents, and
(iv) filling the pH adjusted solution into vials or ampules.

53. The method according to claim 52, whereby the additional active ingredients are selected from the group consisting of non-steroidal anti-inflammatory agents, steroidal anti-inflammatory agents, bronchodilating agents, antirheumatic agents, immunomodulating agents, immunosuppressive agents, corticosteroids, β2-agonists and cholinergic antagonists.

54. The method of claim 1, wherein the neutrophil-dominated, non-infective inflammatory disease is selected from the group consisting of an adult respiratory distress syndrome (ARDS), emphysema, a neutrophil dermatosis, auto-immune bullous dermatoses, vessel-based neutrophilic dermatoses, an autoimmune disease in which neutrophil infiltration is exacerbated by activated complement factors, and an auto-immune disease characterized by acute neutrophil-dominated phases.

55. The method of claim 54, wherein the neutrophil dermatosis is selected from the group consisting of psoriasis and Reiter's syndrome.

56. The method of claim 54, wherein the vessel-based neutrophilic dermatoses disease is selected from the group consisting of leukocytoclastic vasculitis, Sweet's syndrome, pustular vasculitis, erythemanodosum, familial Mediterranean fever, and pyoderma gangrenosum.

57. The method of claim 54, wherein the auto-immune disease in which neutrophil infiltration is exacerbated by activated complement factors, is a renal disease.

58. The method of claim 57, wherein the renal disease is glomerulonephritis.

59. The method of claim 54, wherein the auto-immune disease characterized by acute neutrophil-dominated phases is rheumatoid arthritis.

60. The pharmaceutical composition of claim 40, wherein the neutrophil-dominated, non-infective inflammatory disease is selected from the group consisting of adult respiratory distress syndrome (ARDS), emphysema, a neutrophil dermatosis, auto-immune bullous dermatoses, vessel-based neutrophilic dermatoses, an auto-immune disease in which neutrophil infiltration is exacerbated by activated complement factors, and an autoimmune disease characterized by acute neutrophil-dominated phases.

61. The pharmaceutical composition of claim 60, wherein the neutrophil dermatosis is selected from a group consisting of psoriasis and Reiter's syndrome.

62. The pharmaceutical composition of claim 60, wherein the vessel-based neutrophilic dermatoses is selected from the group consisting of leukocytoclastic vasculitis, Sweet's syndrome, pustular vasculitis, erythema nodosum, familial Mediterranean fever, and pyoderma gangrenosum.

63. The pharmaceutical composition of claim 60, wherein the auto-immune disease in which neutrophil infiltration is exacerbated by activated complement factors, is a renal disease.

64. The pharmaceutical composition of claim 63, wherein the renal disease is glomerulonephritis.

65. The pharmaceutical composition of claim 60, wherein the autoimmune disease characterized by acute neutrophil-dominated phases is rheumatoid arthritis.

66. The method of claim 52, wherein the neutrophil-dominated, non-infective inflammatory disease is selected from the group consisting of adult respiratory distress syndrome (ARDS), emphysema, a neutrophil dermatosis, auto-immune bullous dermatoses, vessel-based neutrophilic dermatoses, an auto-immune disease in which neutrophil infiltration is exacerbated by activated complement factors, and an autoimmune disease characterized by acute neutrophil-dominated phases.

67. The method of claim 66, wherein the neutrophil dermatosis is selected from the group consisting of psoriasis and Reiter's syndrome.

68. The method of claim 66, wherein the vessel-based neutrophilic dermatoses is selected from the group consisting of leukocytoclastic vasculitis, Sweet's syndrome, pustular vasculitis, erythema nodosum, familial Mediterranean fever, and pyoderma gangrenosum.

69. The method of claim 66, wherein the auto-immune disease in which neutrophil infiltration is exacerbated by activated complement factors, is a renal disease.

70. The method of claim 69, wherein the renal disease is glomerulonephritis.

71. The method of claim 66, wherein the autoimmune disease characterized by acute neutrophil-dominated phases is rheumatoid arthritis.
